(19) United States
(12) Patent Application Publication
(10) Pub. No.: US 2025/0325199 A1
(43) Pub. Date: Oct. 23, 2025

(54) Medical Facility Oxygen Monitoring System

(71) Applicant: Ruth Nyobendo, Denver, CO (US)

(72) Inventor: Ruth Nyobendo, Denver, CO (US)

(21) Appl. No.: 18/640,190

(22) Filed: Apr. 19, 2024

(51) Int. Cl. A61B 5/083; A61B 5/00; A61M 16/00; A61M 16/08; A61M 16/10

(52) U.S. Cl. CPC A61B 5/0833; A61B 5/6803; A61B 5/742; A61B 5/746; A61M 16/0003; A61M 16/0051; A61M 16/0875; A61M 16/1005; A61M 2016/003; A61M 2016/1025; A61M 2205/3553; A61M 2205/44

(57) ABSTRACT

A medical facility oxygen monitoring system includes a sensor assembly and a connecting structure detachably connecting said sensor assembly to an oxygen source to permit flow of oxygen gas. The sensor assembly is operatively connectable to an oxygen source and a facial interface to be placed at a face of a patient to direct oxygen into a respiratory system of a patient. The sensor assembly includes a flow meter to monitor flow rate and pressure of oxygen gas, an electronic control system operatively connected to the flow meter to generate an alarm signal when flow rate and pressure indicate uncontrolled flow of oxygen, and a display unit operatively connected to the electronic control system to provide audio or visual alarms to a user.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to oxygen supply systems for hospitals and medical facilities and more particularly pertains to a new medical facility oxygen monitoring system that has a sensor assembly for alerting a user, such as a medical professional, that oxygen is flowing in an uncontrolled manner, such as when an oxygen mask or nasal cannula has been removed from the patient or disconnected from the sensor assembly but the oxygen flow has not been turned off. The alarm will alert the user to turn off the flow of oxygen and thereby avoid wasting oxygen.

### (2) Description of Related Art Including Information Disclosed Under 37 CFR 1.97 and 1.98

The prior art relates to oxygen supply systems for hospitals and medical facilities. The prior art, as best understood, does not disclose an oxygen monitoring system that includes a sensor assembly to detect and alert a user when oxygen is flowing in an uncontrolled manner, such as when an oxygen mask or nasal cannula has been removed from the patient or disconnected from the sensor assembly.

### BRIEF SUMMARY OF THE INVENTION

An embodiment of the disclosure meets the needs presented above in a medical facility oxygen monitoring system generally comprising a sensor assembly and a connecting structure detachably connecting said sensor assembly to an oxygen source to permit flow of oxygen gas. The sensor assembly is operatively connectable to an oxygen source and a facial interface to be placed at a face of a patient to direct oxygen into a respiratory system of a patient. The sensor assembly includes a flow meter to monitor flow rate and pressure of oxygen gas, an electronic control system operatively connected to the flow meter to generate an alarm signal when flow rate and pressure indicate uncontrolled flow of oxygen, and a display unit operatively connected to the electronic control system to provide audio or visual alarms to a user. In operation, when the facial interface device, such as an oxygen mask or nasal cannula, is removed from the patient or disconnected from the sensor assembly but the flow of oxygen is not turned off, the sensor assembly detects this change in flow rate and pressure and generates an alarm signal so that a user, such as a medical professional, can turn off the flow of oxygen and thereby avoid wasting oxygen.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a side view of a medical facility oxygen monitoring system according to an embodiment of the disclosure in use.

FIG. 2 is a side view of an embodiment of the disclosure in use.

FIG. 3 is a block diagram showing the relationship of components of an embodiment of the disclosure.

FIG. 4 is a side view of an alternative embodiment of the disclosure in use.

FIG. 5 is a block diagram showing the relationship of components of an alternative embodiment of the disclosure.

FIG. 6 is a side view of an alternative embodiment of the disclosure in use.

### DETAILED DESCRIPTION OF THE INVENTION

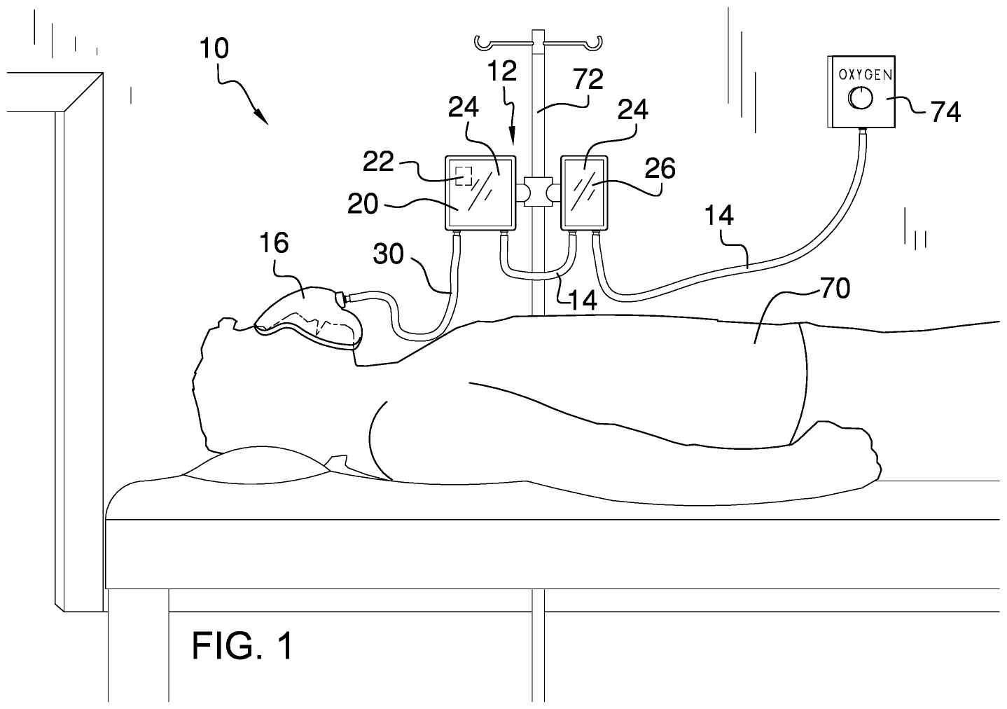

With reference now to the drawings, and in particular to FIGS. 1 through 6 thereof, a new medical facility oxygen monitoring system embodying the principles and concepts of an embodiment of the disclosure and generally designated by the reference numeral 10 will be described.

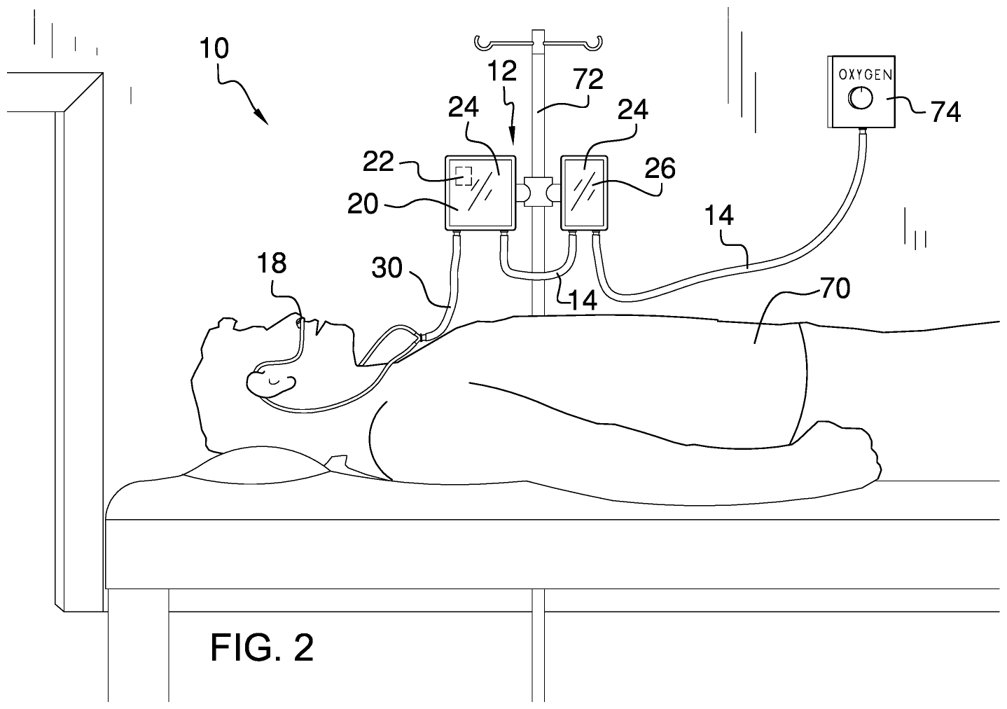

As best illustrated in FIGS. 1 through 6, the medical facility oxygen monitoring system 10 generally comprises a sensor assembly 12 and a connecting structure 14 detachably connecting said sensor assembly 12 to an oxygen source to permit flow of oxygen gas. The sensor assembly 12 is operatively connectable to an oxygen source and a facial interface, such as an oxygen mask 16 shown in FIG. 1 or nasal cannula 18 shown in FIG. 2, to be placed at a face of a patient 70 to direct oxygen into a respiratory system of the patient 70. The sensor assembly 12 includes a flow meter 20 to monitor flow rate and pressure of oxygen gas, an electronic control system 22 operatively connected to the flow meter 20 to generate an alarm signal when flow rate and pressure indicate uncontrolled flow of oxygen, and a display unit 24 operatively connected to the electronic control system 22 to provide audio or visual alarms to a user. As shown in FIGS. 1 and 2, the sensor assembly 12 also can include an oxygen flow sensor 26 operatively connected to the electronic control system 22. The medical facility oxygen monitoring system 10 includes an additional connecting structure 14 detachably connecting the flow meter 20 and the oxygen flow sensor 26, wherein the additional connecting structure 14 includes tubing.

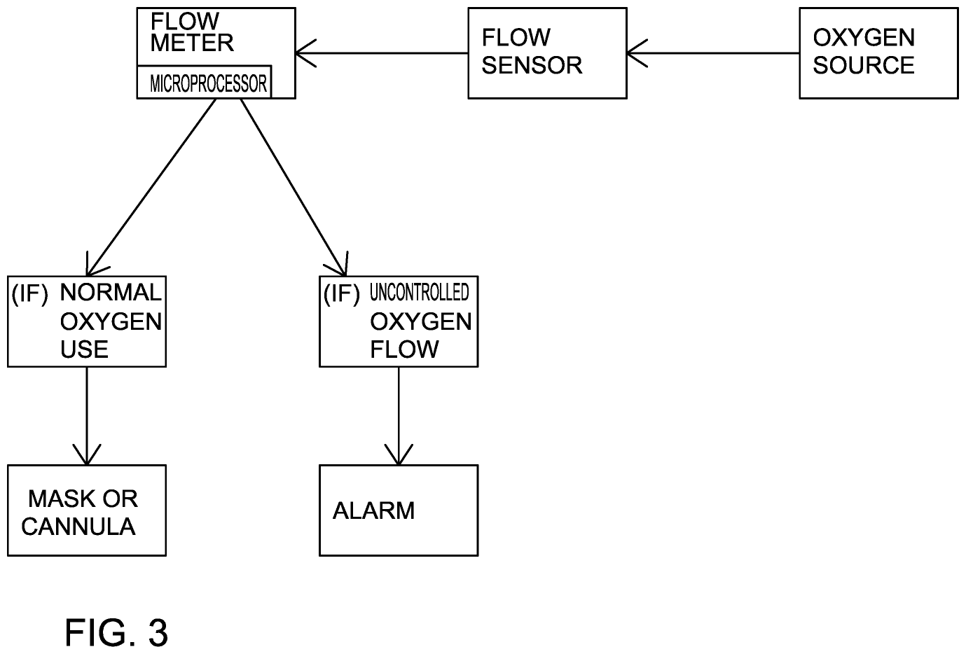

As shown in FIG. 3, when using the medical facility oxygen monitoring system 10, oxygen flows from the oxygen source to the flow sensor 26 and then to the flow meter 20. A microprocessor of the electronic control system 22 receives the sensor information. If normal oxygen use is detected, then oxygen flows normally to the mask 16 or nasal cannula 18 via tubing 30. However, if a pressure or flow change is detected evidencing an uncontrolled flow of oxygen, such as if the patient 70 is no longer wearing the mask 16 or nasal cannula 18 or if the connecting tubing is disconnected from the flow meter 20, then an alarm signal is generated. The display unit 24, which could be a display screen, will then display a visual alarm message or light. The display unit 24 could also include speakers or similar sound producing device to produce an audio alarm, such as a beeping sound. In this manner a user, such as a medical professional or staff, would be alerted that oxygen is flowing in an uncontrolled and wasteful manner and should be shut off.

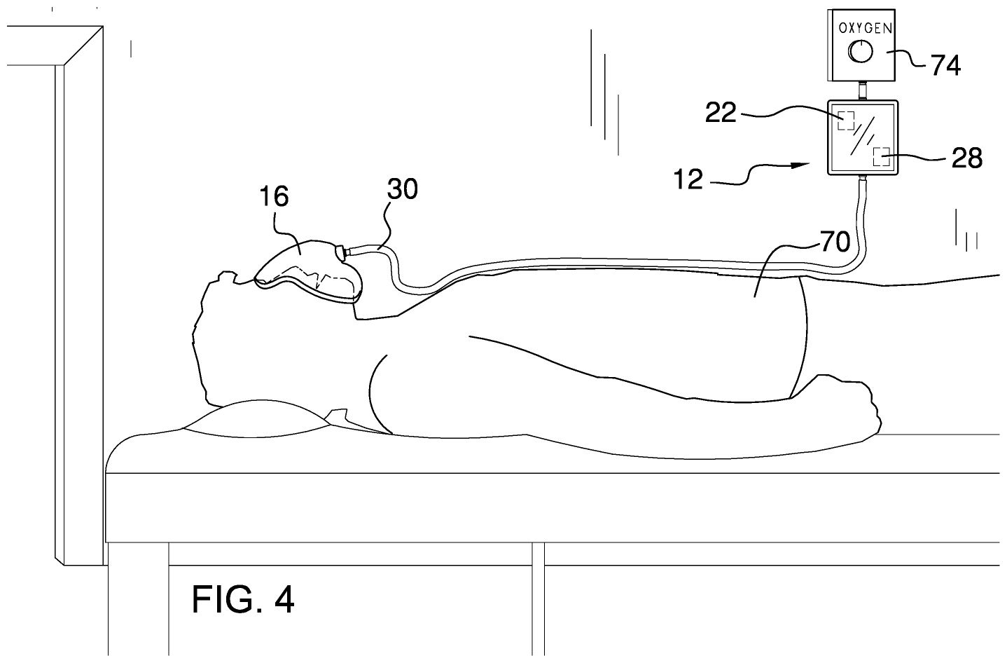

In one possible embodiment shown in FIG. 4, the sensor assembly 12 includes a time sensor 28 to detect duration of flow of oxygen gas. The time sensor 28 is operatively connected to the electronic control system 22. The electronic control system 22 is designed to generate an audio or visual alarm signal when duration of flow of oxygen exceeds a selected duration. In use, the time sensor 28 could be set to a particular time duration. For example, if the patient 70 is to use oxygen overnight while sleeping, the time sensor 28 could be set to a normal amount of sleep, such as six to eight hours. When that time expires, the electronic control system 22 sends out an alarm signal to remind a user, such as medical or hospital staff, to check the oxygen system. In this manner the staff can be reminded to make sure that the oxygen flow has been shut off after the end of normal usage during sleep by the patient 70. Another possibility would be to set the timer at a time that is beyond any expected or normal use, such as 16-24 hours. This way if the oxygen is still flowing for a period of time that is very likely beyond any normal use, then it is likely that the oxygen has been left on unnecessarily and should be shut off. While some oxygen would be wasted in this scenario, excessive loss of oxygen over several days could be avoided.

As shown in FIGS. 1 and 2, the medical facility oxygen monitoring system 10 further includes a support frame attached to the sensor assembly 12 for mounting the sensor assembly 12 on a stand, such as a wheeled stand 72 normally used for hanging medical bags or other devices. Alternatively, as shown in FIG. 4, the sensor assembly 12 is detachably mountable to a wall outlet 74 of an oxygen source. The sensor assembly 12 could remain mounted to the wall outlet 74 for repeated use as an essentially permanent component of the oxygen system, wherein the connecting tubing of a mask 16 or nasal cannula 18 could be detachably connected to the sensor assembly 12.

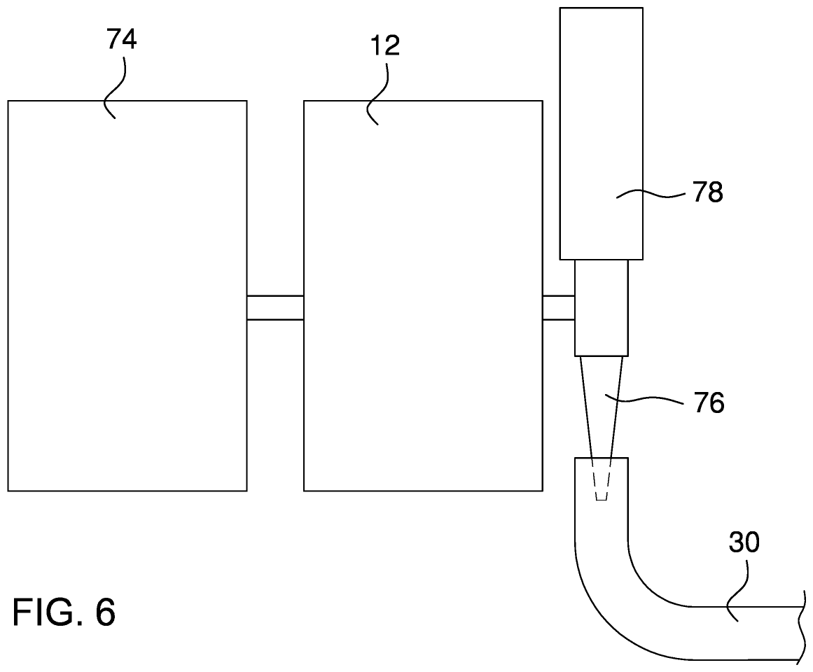

FIG. 6 shows one possible embodiment in which the sensor assembly 12 is attached, either permanently or by a detachable connection, to a wall outlet 74 for oxygen. A standard oxygen regulator valve assembly 78 is attached to the sensor assembly 12, also either permanently or by a detachable connection. A valve connector known as a christmas tree 76 is removably attached to the oxygen regulator valve assembly 78. A hose or tubing 30 that is part of the oxygen mask 16 or nasal cannula 18 is connected to the christmas tree 76. Whenever the oxygen mask 16 or nasal cannula 18 is disconnected from the patient, or whenever the hose or tubing 30 is disconnected from the christmas tree 76, this change in flow or flow pressure or flow rate is detected by the sensor assembly 12 and an alert is generated as discussed above to notify personnel that unregulated flow of oxygen is occurring.

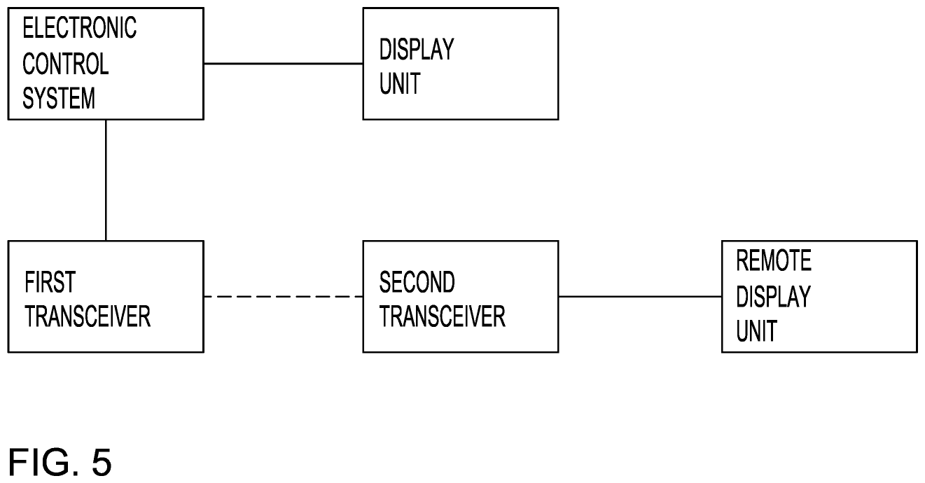

The medical facility oxygen monitoring system 10 could also include a remote display unit that is wirelessly connected to the electronic control system 22. FIG. 5 shows a block diagram, wherein the electronic control system 22 is directly connected to the display unit 24, such as shown in FIGS. 1 and 2, but also connected by a first transceiver and a second transceiver to a remote display unit located in a different location than the electronic control system 22, such as at a nurse station in a hospital. This would allow a user to remotely monitor the oxygen system 10.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A medical facility oxygen monitoring system comprising:
a sensor assembly operatively connectable to an oxygen source and a facial interface to be placed at a face of a patient to direct oxygen into a respiratory system of a patient, wherein said sensor assembly comprises:
a flow meter to monitor flow rate and pressure of oxygen gas,
an electronic control system operatively connected to said flow meter to generate an alarm signal when flow rate and pressure indicate uncontrolled flow of oxygen, and
a display unit operatively connected to said electronic control system to provide audio or visual alarms to a user; and
a connecting structure detachably connecting said sensor assembly to an oxygen source to permit flow of oxygen gas.

2. The medical facility oxygen monitoring system of claim 1, wherein said connecting structure comprises tubing.

3. The medical facility oxygen monitoring system of claim 2, wherein said sensor assembly comprises an oxygen flow sensor operatively connected to said electronic control system, and the medical facility oxygen monitoring system comprises an additional connecting structure detachably connecting said flow meter and said oxygen flow sensor, wherein said additional connecting structure comprises tubing.

4. The medical facility oxygen monitoring system of claim 3, wherein said sensor assembly comprises a time sensor to detect duration of flow of oxygen gas, said time sensor is operatively connected to said electronic control system, and said electronic control system is configured to generate an audio or visual alarm signal when duration of flow of oxygen exceeds a selected duration.

5. The medical facility oxygen monitoring system of claim 4, wherein the medical facility oxygen monitoring system further comprises a support frame attached to said sensor assembly for mounting on a stand.

6. The medical facility oxygen monitoring system of claim 4, wherein said sensor assembly is detachably mountable to a wall outlet of an oxygen source.

7. The medical facility oxygen monitoring system of claim 4, wherein the medical facility oxygen monitoring system further comprises a remote display unit wirelessly connected to said electronic control system.

8. The medical facility oxygen monitoring system of claim 1, wherein said sensor assembly comprises an oxygen flow sensor operatively connected to said electronic control system, and the medical facility oxygen monitoring system comprises an additional connecting structure detachably connecting said flow meter and said oxygen flow sensor, wherein said additional connecting structure comprises tubing.

9. The medical facility oxygen monitoring system of claim 1, wherein said sensor assembly comprises a time sensor to detect duration of flow of oxygen gas, said time sensor is operatively connected to said electronic control system, and said electronic control system is configured to generate an audio or visual alarm signal when duration of flow of oxygen exceeds a selected duration.

10. The medical facility oxygen monitoring system of claim 1, wherein the medical facility oxygen monitoring system further comprises a support frame attached to said sensor assembly for mounting on a stand.

11. The medical facility oxygen monitoring system of claim 1, wherein said sensor assembly is detachably mountable to a wall outlet of an oxygen source.

12. The medical facility oxygen monitoring system of claim 1, wherein the medical facility oxygen monitoring system further comprises a remote display unit wirelessly connected to said electronic control system.
